(19) United States
(12) Patent Application Publication
(10) Pub. No.: US 2026/0015590 A1
(43) Pub. Date: Jan. 15, 2026

(54) METHODS OF PRODUCING RECOMBINANT ADENOVIRUS VECTORS

(71) Applicant: Ferring International Center S.A., Saint-Prex (CH)

(72) Inventor: Florian Madura, Saint-Prex (CH)

(21) Appl. No.: 18/769,686

(22) Filed: Jul. 11, 2024

(51) Int. Cl. C12N 7/00; C07K 14/56; C12N 5/071

(52) U.S. Cl. CPC C12N 7/00; C07K 14/56; C12N 5/0686; C12N 2710/10043; C12N 2710/10051

(57) ABSTRACT

Disclosed herein are methods for producing recombinant adenoviruses or recombinant adenovirus vectors.

### RELATED APPLICATION INFORMATION

None.

### TECHNICAL FIELD

The present disclosure relates to methods for producing recombinant adenoviruses or recombinant adenovirus vectors.

### BACKGROUND

Recombinant adenovirus vectors have emerged as a potent therapeutic means to treat several diseases, including a variety of cancers. For example, Nadofaragene firadenovec (also known as ADSTILADRIN®), is a gene therapy product approved by the U.S. Food and Drug Administration for the treatment of adult patients with high-risk Bacillus Calmette-Guérin (BCG)-unresponsive non-muscle invasive bladder cancer (NMIBC) with carcinoma in situ (CIS) with or without papillary tumors. Such gene therapy vector has proven to be effective to treat subjects in need of treatment. Improved methods of producing high-quality adenovirus vectors are needed to accelerate patient access to these innovative therapies. The present disclosure provides improved methods to produce recombinant adenovirus vectors in amounts required to meet increasing clinical demand.

### SUMMARY

In one embodiment, the present disclosure relates to a method of manufacturing an adenovirus. In some embodiment, the method comprises the steps of:
a) infecting Viral Production Cells 2.0 (VPC2.0 cells) with at least one non-replicating recombinant adenovirus vector containing a transgene encoding human interferon; and
b) harvesting the vector containing the transgene encoding human interferon produced by the VPC2.0 cells.

In some embodiments of the above method, the interferon is interferon alpha. In yet further embodiments, the interferon alpha is interferon alpha-2b.

In still other embodiments of the above method, the non-replicating vector is a non-replicating recombinant adenovirus serotype 5 (Ad5) vector. In still yet further embodiments, the Ad5 vector is Nadofaragene firadenovec (also known as ADSTILADRIN®).

In still other embodiments of the above method, the vector is harvested at least 24 hours after infection. In still yet further embodiments, the vector is harvested at least 36 hours after infection. In even still further embodiments, the vector is harvested about 48 hours after infection.

In still yet further embodiments of the above method, the VPC2.0 cells are cultured in suspension.

In other embodiments of the above method, the multiplicity of infection of the vector to VPC2.0 cells is about 100 to about 300. In still other embodiments, the multiplicity of infection of the vector to VPC2.0 cells is about 175 to about 200.

In still other embodiments of the above method, the VPC2.0 cells are inoculated in a bioreactor at a target seeding cell density of about 0.75×10⁶ viable cells/mL. In still other embodiments, the VPC2.0 cells are infected at a target infection cell density of from about 2×10⁶ to about 4×10⁶ viable cells/mL.

In yet further embodiments of the above method, when the vector is Nadofaragene firadenovec, the Nadofaragene firadenovec is produced in an amount at least 100 percent (%) greater than a method in which HEK293 cells (e.g., which is not VPC2.0 cells) are infected with Nadofaragene firadenovec. In still other embodiments, when the vector is Nadofaragene firadenovec, the Nadofaragene firadenovec is produced in an amount at least 200% greater, at least 300% greater, at least 400% greater, or at least 500% greater than a method in which HEK293 (e.g., which is not VPC2.0 cells) are infected with Nadofaragene firadenovec.

In still other embodiments of the above method, the method further comprises lysing the VPC2.0 cells with a lysing agent prior to harvesting. In still other embodiments of the above method, the method further comprises centrifuging the lysed cells.

In still other embodiments of the above method, the method further comprises purifying the vector after harvesting.

In another embodiment, the present disclosure relates a method of manufacturing an adenovirus comprising the steps of:
a) infecting HEK293 cells grown in suspension with at least one non-replicating recombinant adenovirus vector containing a transgene encoding human interferon, wherein the target infection cell density is from about 1×10⁶ to about 4×10⁶ viable cells/mL, and infection is carried out at a multiplicity of infection of about 100 to about 300 and a duration of infection is about 48 hours; and
b) harvesting the vector containing the transgene encoding human interferon produced by the HEK293 cells.

In some embodiments of the above method, the cells are a clonal cell line derived from HEK293F cells. In still other embodiments, the clonal cell line is Viral Production Cells 2.0 (VPC2.0).

In some embodiments of the above method, the interferon is interferon alpha. In yet further embodiments, the interferon alpha is interferon alpha-2b.

In still other embodiments of the above method, the non-replicating vector is a non-replicating adenovirus serotype 5 (Ad5) vector. In still yet further embodiments, the Ad5 vector is Nadofaragene firadenovec (also known as ADSTILADRIN®).

In still other embodiments of the above method, the vector is harvested at least 24 hours after infection. In still yet further embodiments, the vector is harvested at least 36 hours after infection. In even still further embodiments, the vector is harvested about 48 hours after infection.

In other embodiments of the above method, the multiplicity of infection of the vector to VPC2.0 cells is about 100 to about 300. In still other embodiments, the multiplicity of infection of the vector to VPC2.0 cells is about 175 to about 200.

In still other embodiments of the above method, the VPC2.0 cells are inoculated in a bioreactor at a target seeding cell density of about 0.1 to about 10×10⁶ viable cells/mL. In still other embodiments, the VPC2.0 cells are inoculated in a bioreactor at a target seeding cell density of about 0.75×10⁶ viable cells/mL. In still other embodiments, the VPC2.0 cells are infected at a target infection cell density of from about 2×10⁶ to about 4×10⁶ viable cells/mL.

In yet further embodiments of the above method, when the vector is Nadofaragene firadenovec, the Nadofaragene firadenovec is produced in an amount at least 100 percent (%) greater than a method in which HEK293 cells (e.g., namely ATCC Accession No. CRL-1573 which is not VPC2.0 cells) are infected with Nadofaragene firadenovec. In still other embodiments, when the vector is Nadofaragene firadenovec, the Nadofaragene firadenovec is produced in an amount at least 200% greater, at least 300% greater, at least 400% greater, or at least 500% than a method in which HEK293 (e.g., namely, ATCC Accession No. CRL-1573 which is not VPC2.0 cells) are infected with Nadofaragene firadenovec.

In still other embodiments of the above method, the method further comprises lysing the VPC2.0 cells with a lysing agent prior to harvesting. In still other embodiments of the above method, the method further comprises centrifuging the lysed cells.

In still other embodiments of the above method, the method further comprises purifying the vector after harvesting.

### BRIEF DESCRIPTION OF THE DRAWINGS

Having thus described the presently disclosed subject matter in general terms, reference will now be made to the accompanying FIGURES, which are not necessarily drawn to scale, and wherein:

FIG. 1 shows the estimated number of doses of rAd-IFN (ADSTILADRIN®) produced using VPC2.0 cells compared to other methods known in the art as described in Example 1.

### DETAILED DESCRIPTION

In some embodiments, the present disclosure relates to a new method for preparing large scale quantities of a recombinant adenovirus or recombinant adenovirus vector. In other embodiments, when the methods described herein are used to manufacture Nadofaragene firadenovec, the amount of Nadofaragene firadenovec produced is between about 100% to about 575% greater than methods known in the prior art. This is highly desirable, as there are currently no techniques available to produce the very large, commercial quantities of Nadofaragene firadenovec required for clinical applications, such as in the use of treating patients suffering from cancer, particularly, bladder cancer.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “or” means “and/or” unless stated otherwise. Furthermore, the use of the terms “including” and “having,” as well as other forms of those terms, such as “includes,” “included”, “has,” and “have” are not limiting.

The term “about” is utilized herein to specify precise numerical support for the value it precedes, as well as to encompass numbers in close proximity to or approximating the specified value. Certain ranges are delineated in this document with numerical values preceded by the term “about.” When determining if a number is near to or approximates a specifically stated value, the unrecited number that closely approximates the stated value, considering the context in which it is presented, may be deemed substantially equivalent to the specified number.

In instances where a range of values is provided, it is understood that every value within that range, to the nearest tenth of the unit of the lower limit unless otherwise indicated by the context, including any other stated or intervening values within that range, falls within the scope of the invention. Both the upper and lower limits of these smaller ranges may be included independently within the smaller ranges and are also considered part of the invention, except where explicitly excluded by stated limits within the range. If the stated range includes one or both of its limits, ranges excluding either or both of these included limits are also considered part of the invention.

As used herein, the term “HEK293” refers to immortalized human embryonic kidney cells. HEK293 was originally isolated in the 1970's by Alex Van der Eb, a Dutch biologist, and it was his postdoc, Frank Graham who transformed the cell line with a sheared adenovirus 5 (Ad5). The ‘293’ refers to the fact that it was Graham's 293rd experiment. HEK293 has been deposited at the American Type Culture Collection under Accession No. CRL-1573. Several variants of HEK293 are known, including HEK293S, HEK293T, HEK293F, HEK293FT, HEK293FTM, HEK293SG, HEK293SF, HEK293STF, etc. For example, HEK293F cells are a polyclonal isolate from the HEK293 cell line adapted to specific culture conditions. Specifically, HEK293F cells have been adapted to suspension culture in the serum-free media, such as the 293 SFM II media. HEK293F cells are commercially available from ThermoFisher Scientific (Waltham, MA).

As used herein the phrase “multiplicity of infection” or “MOI” as used interchangeably herein, refers to the number of virions that are added per cell during infection. For example, if one million virions are added to one million cells, the MOI is one. If ten million virions are added to one million cells, the MOI is ten.

As used herein, the phrase “Nadofaragene firadenovec”, “ADSTILADRIN®”, or “rAd-IFN” as used interchangeably herein refers to recombinant adenovirus serotype 5 (Ad5) vector containing a transgene encoding the human interferon alfa-2b (IFNα2b). Nadofaragene 1 firadenovec has been approved by the U.S. Food and Drug Administration for the treatment of adult patients with high-risk Bacillus Calmette-Guérin (BCG)-unresponsive non-muscle invasive bladder cancer (NMIBC) with carcinoma in situ (CIS) with or without papillary tumors. The recommended dose is 75 mL at a concentration of 3×10¹¹ viral particles (vp)/mL instilled once every three months into the bladder via a urinary catheter.

As used herein, the term “recombinant cell” refers to a cell into which a gene or transgene, such as a gene or transgene from the adenoviral genome or from another cell, has been introduced. Recombinant cells are distinguishable from naturally occurring cells which do not contain a recombinantly-introduced gene. Recombinant cells are thus cells having a gene or genes introduced through “the hand of man.”

As used herein, the phrase “Viral Production Cells 2.0” or “VPC2.0 cells”, as used interchangeably herein refers to a clonal cell line derived from HEK293F. VPC2.0 cells are available from ThermoFisher Scientific (Waltham, MA) (Catalog number A49784).

### Methods for Manufacturing a Recombinant Adenovirus or Adenoviral Vector

Production of recombinant adenoviruses or adenovirus vectors, as used interchangeably herein, have been described previously, such as, for example, in WO 2016/048556. The present disclosure relates to new methods for preparing large scale quantities of a recombinant adenovirus or recombinant adenovirus vector, particularly Nadofaragene firadenovec. As will be described in further detail herein, the methods involve infecting suspension grown HEK293 cells with at least one non-replicating recombinant adenovirus or recombinant adenoviral vector containing at least one transgene encoding human interferon (e.g., such as in a bioreactor) and then harvesting the vector containing the transgene encoding human interferon. In some embodiments, the infection of the HEK293 cells with at least one non-replicating adenovirus or adenoviral vector occurs in a bioreactor.

Many therapeutic adenoviral vectors, such as recombinant Ad5 (rAd5) vectors, are non-replicating (or replication deficient) in which the genome is deleted in the E1 region to provide space for alternate gene expression cassettes. The E1 region encodes proteins necessary for the expression of the other early and late genes, hence initiating the viral life cycle. Thus, when the E1 region is replaced with an expression cassette to produce the transgene that is useful in therapy, such as, for example, interferons, cytokines, suicide genes, antigens or antibodies, a producer cell line containing adenovirus E1 sequences is required to complement for this region. Therefore, in those embodiments in which the recombinant adenovirus vector lacks the adenovirus E1 region, the HEK293 cells described herein will contain adenovirus E1 sequences.

Methods for constructing non-replicating (or replication deficient) recombinant adenoviruses or recombinant adenovirus vectors containing one or more transgenes are well known in the art and involve the use of standard molecular biological techniques, such as those described in, for example, Sambrook et al., Molecular Cloning, a Laboratory Manual, 2d ed., Cold Spring Harbor Press, Cold Spring Harbor, N.Y. (1989), Watson et al., Recombinant DNA, 2d ed., Scientific American Books (1992), and Ausubel et al., Current Protocols in Molecular Biology, Wiley Interscience Publishers, NY (1995).

In some embodiments, the transgene contained in the non-replicating recombinant adenovirus or recombinant adenovirus vector is any transgene that encodes a human interferon. In some embodiments, the transgene that encodes human interferon encodes interferon alpha, such as, for example, interferon alpha-2b. In other embodiments, the transgene that encodes human interferon encodes interferon beta. In still other embodiments, the transgene that encodes interferon encodes interferon gamma.

In some embodiments, the non-replicating vector is a non-replicating recombinant adenovirus serotype (5) (Ad5) vector. In still other embodiments, the Ad5 vector is Nadofaragene firadenovec.

As mentioned previously herein, in one embodiment, the present disclosure relates to a method of manufacturing a recombinant adenovirus or recombinant adenovirus vector which involves infecting HEK293 cells able to grow in suspension with at least one non-replicating recombinant adenovirus or recombinant adenovirus vector containing at least one transgene encoding human interferon. The HEK293 cells can be infected with the at least one non-replicating adenovirus containing the at least one transgene encoding human interferon, using routine techniques known in the art.

In some embodiments, the infection is carried out at a target infection cell density from about 1.0×10⁶ to about 5.0×10⁶ viable cells/mL. In some embodiments, the infection is carried out at a target infection cell density from about 2.0×10⁶ to about 4.0×10⁶ viable cells/mL.

In some other embodiments, the infection is carried out at a multiplicity of infection (MOI) of about 100 to about 300, such as a MOI of about 150 to about 250. In some embodiments, the infection is carried out at a MOI of about 200.

In yet some other embodiments the duration of infection (DOI) is about 32 hours to about 72 hours. In some embodiments, the DOI is about 48 hours.

In some embodiments, the method comprises: (a) infecting the cells at a target infection cell density from about 1.0×10⁶ to about 5.0×10⁶ viable cells/mL, in particular from about 2.0×10⁶ to about 4.0×10⁶ viable cells/mL; (b) infecting the cells at a MOI of about 100 to about 300, such as a MOI of about 150 to about 250, in particular at a MOI of about 200; and (c) infecting the cells for a DOI about 32 hours to about 72 hours, such as infecting the cells for a DOI of about 48 hours.

In some embodiments, the HEK293 cells are a polyclonal or a monoclonal cell line derived from HEK293F cells. In a particular embodiment, the HEK293 cells are polyclonal cells derived from HEK293F such as the Viral Production Cells (available from ThermoFisher Scientific (Waltham, MA); Catalog number A35347). In still further embodiments, the HEK293 cells are monoclonal cells derived from HEK293F cells. In a further particular embodiment, the monoclonal cells are Viral Production Cells 2.0 (VPC2.0).

In still yet other embodiments, the methods comprise infecting VPC2.0 cells with at least one non-replicating recombinant adenovirus or recombinant adenovirus vector containing at least one transgene encoding human interferon. In some embodiments, the non-replicating virus or non-replicating vector is a non-replicating, recombinant Ad5 vector. In yet still other embodiments, the recombinant Ad5 vector is Nadofaragene firadenovec.

In some embodiments, the HEK293 cells used in the method are seeded or grown in a cell culture medium (e.g., cell culture) prior to infection with the at least one non-replicating recombinant adenovirus or recombinant adenoviral vector. The cell culture medium used can be any cell culture medium known in the art, such as a chemically defined medium, a serum-free medium, a protein-free medium, or any combination thereof. A chemically defined medium is a medium which contains components the exact concentration of which is known, such as a basal medium (e.g. DMEM, F12 or RPMI), amino acids, vitamins, inorganic salts, buffers, antioxidants and energy source (such as glucose, L-glutamine and/or L-alanyl-L-glutamine). A chemically defined medium may be further supplemented with recombinant proteins or peptides, such as recombinant albumin, recombinant insulin. Such chemically defined media are well known in the art. For HEK293 cells, examples of suitable media include, but are not limited to, CD 293 medium, BHK, Ex-Cell medium, Freestyle, Hyclone SFM293 culture media, the Gibco™ Viral Production Medium, AAV-Max media (available from ThermoFisher Scientific (Waltham, MA), or CTS AAV-MAX media (Gibco; Fisher Scientific (Waltham, MA)).

In some embodiments, the recombinant adenovirus or recombinant adenovirus vector is manufactured in a bioreactor. In further embodiments, when a bioreactor is to be used to manufacture the recombinant adenovirus or recombinant adenovirus vector, the HEK293 cells from a working cell bank can be expanded, prior to infection. Expansion in suspension mode can occur using any cell culture medium known in the art, such as a chemically defined medium, a serum-free medium, a protein-free medium, or any combination thereof, such as those described previously herein.

Expansion of the infected HEK293 cells can take place using any techniques known in the art. For example, expansion may be started in suspension using one or more culture vessels, such as one or more flasks, such as, for example T-flask(s) or roller bottle(s) and followed by a proliferation period in other flasks or roller bottles until a sufficient number of cells is reached to further expand the cells. In some embodiments, once a sufficient quantity of cells is reached, further expansion can be undertaken in a larger scale system, such as in a batch, fed batch, or perfusion system until a sufficient number of cells is reached for inoculation into a bioreactor.

Once expansion is complete, the HEK293 cells can be inoculated into a bioreactor. The type of bioreactor used to produce the recombinant adenovirus or recombinant adenoviral vector is not critical and are well known in the art. In some embodiments, the bioreactor is a bioreactor suitable for batch, fed batch or perfusion culture modes. In some embodiments, inoculation into a bioreactor is performed to achieve a target seeding cell density of HEK293 cells of about 0.1×10⁶ to about 5×10⁶ viable cells/mL. In some embodiments, inoculation into a bioreactor is performed to achieve a target seeding cell density of HEK293 cells of about 0.75×10⁶ viable cells/mL.

In still other embodiments, prior to inoculation, the HEK293 cells may be centrifuged to obtain a cell pellet and suspended in an appropriate volume of growth medium for inoculation. In some embodiments, the cells are inoculated into the bioreactor directly from expansion culture.

Once inoculated into a bioreactor, the HEK293 cells can be expanded for a period of time to allow for a suitable cell density to be reached to allow for the production of clinical or commercial batches of the recombinant adenovirus or adenovirus vector. In some embodiments, the HEK293 cells are expanded in the bioreactor for a period of time of about 12 to about 72 hours after inoculation and before infection, such as for a period of time of about 24 to about 48 hours, until they reach the target infection cell density.

After the HEK293 cells are inoculated into a bioreactor and a suitable cell density is reached, the cells are infected with at least one non-replicating recombinant adenovirus or adenovirus vector containing a transgene encoding human interferon to be propagated. In some embodiments, the HEK293 cells are infected with the non-replicating adenovirus or adenovirus vector during or after late-log phase of growth and before stationary phase. As used herein, “late-log phase” refers to cell growth approaching the end of logarithmic growth, and before reaching the stationary phase of growth. Late-log phase can typically be identified on a growth curve as a secondary or tertiary point of inflection that occurs as the log-growth phase slows, approaching stationary growth.

The pH in the bioreactor is maintained between 7.0 to 7.4. In some embodiments, the pH in the bioreactor is maintained at 7.0. In other embodiments, the pH in the bioreactor is maintained at 7.1. In still other embodiments, the pH in the bioreactor is maintained at 7.2. In still other embodiments, the pH in the bioreactor is maintained at 7.3. In still other embodiments, the pH in the bioreactor is maintained at 7.4.

Infection of the HEK293 cells with the at least one non-replicating recombinant adenovirus vector containing a transgene encoding human interferon is performed at a MOI of about 50 to about 300. In some embodiments, the MOI is from about 50 to about 250. In yet other embodiments, the MOI is from about 50 to about 150. In some embodiments, the MOI is from about 100 to about 275. In other embodiments, the MOI is from about 100 to about 250. In other embodiments, the MOI is from about 100 to about 200. In still other embodiments, the MOI is from about 100 to about 175. In still other embodiments, the MOI is from about 100 to about 150. In further embodiments, the MOI is about 50. In still further embodiments, the MOI is about 60. In still further embodiments, the MOI is about 70. In still further embodiments, the MOI is about 80. In still further embodiments, the MOI is about 90. In still further embodiments, the MOI is about 100. In still other embodiments, the MOI is about 110. In still further embodiments, the MOI is about 120. In yet further embodiments, the MOI is about 130. In yet further embodiments, the MOI is about 140. In still further embodiments, the MOI is about 150. In yet further embodiments, the MOI is about 160. In yet further embodiments, the MOI is about 170. In still further embodiments, the MOI is about 180. In yet further embodiments, the MOI is about 190. In yet still further embodiments, the MOI is about 200. In still other embodiments, the MOI is about 210. In still further embodiments, the MOI is about 220. In yet further embodiments, the MOI is about 230. In yet further embodiments, the MOI is about 240. In still further embodiments, the MOI is about 250. In yet further embodiments, the MOI is about 260. In yet further embodiments, the MOI is about 270. In still further embodiments, the MOI is about 280. In yet further embodiments, the MOI is about 290. In yet still further embodiments, the MOI is about 300. In a particular embodiment, the MOI is of about 200.

Additionally, in some embodiments, the target infection cell density is from about 0.5×10⁶ to about 5.0×10⁶ viable cells/mL. In some embodiments, the target infection cell density is from about 0.5×10⁶ to about 4.0×10⁶ viable cells/mL. In some embodiments, the target infection cell density is about 0.5×10⁶ viable cells/mL. In some embodiments, the target infection cell density is about 0.6×10⁶ viable cells/mL. In some embodiments, the target infection cell density is about 0.7×10⁶ viable cells/mL. In some embodiments, the target infection cell density is about 0.8×10⁶ viable cells/mL. In some embodiments, the target infection cell density is about 0.9×10⁶ viable cells/mL. In some embodiments, the target infection cell density is about 1.0×10⁶ viable cells/mL. In some embodiments, the target infection cell density is about 1.1×10⁶ viable cells/mL. In still other embodiments, the target infection cell density is about 1.2×10⁶ viable cells/mL. In still other embodiments, the target infection cell density is about 1.3×10⁶ viable cells/mL. In still other embodiments, the target infection cell density is about 1.4×10⁶ viable cells/mL. In still other embodiments, the target infection cell density is about 1.5×10⁶ viable cells/mL. In still other embodiments, the target infection cell density is about 1.6×10⁶ viable cells/mL. In still other embodiments, the target infection cell density is about 1.7×10⁶ viable cells/mL. In still other embodiments, the target infection cell density is about 1.8×10⁶ viable cells/mL. In still other embodiments, the target infection cell density is about 1.9×10⁶ viable cells/mL. In some embodiments, the target infection cell density is about 2.0×10⁶ viable cells/mL. In some embodiments, the target infection cell density is about 2.1×10⁶ viable cells/mL. In still other embodiments, the target infection cell density is about 2.2×10⁶ viable cells/mL. In still other embodiments, the target infection cell density is about 2.3×10⁶ viable cells/mL. In still other embodiments, the target infection cell density is about 2.4×10⁶ viable cells/mL. In still other embodiments, the target infection cell density is about 2.5×10⁶ viable cells/mL. In still other embodiments, the target infection cell density is about 2.6×10⁶ viable cells/mL. In still other embodiments, the target infection cell density is about 2.7×10⁶ viable cells/mL. In still other embodiments, the target infection cell density is about 2.8×10⁶ viable cells/mL. In still other embodiments, the target infection cell density is about 2.9×10⁶ viable cells/mL. In still other embodiments, the target infection cell density is about 3.0×10⁶ viable cells/mL. In still other embodiments, the target infection cell density is about 3.1×10⁶ viable cells/mL. In still other embodiments, the target infection cell density of about 3.2×10⁶ viable cells/mL. In still other embodiments, the target infection cell density is about 3.3×10⁶ viable cells/mL. In still other embodiments, the target infection cell density is about 3.4×10⁶ viable cells/mL. In still other embodiments, the target infection cell density is about 3.5×10⁶ viable cells/mL. In still other embodiments, the target infection cell density is about 3.6×10⁶ viable cells/mL. In still other embodiments, the target infection cell density is about 3.7×10⁶ viable cells/mL. In still other embodiments, the target infection cell density is about 3.8×10⁶ viable cells/mL. In still other embodiments, the target infection cell density is about 3.9×10⁶ viable cells/mL. In still other embodiments, the target infection cell density is about 4.0×10⁶ viable cells/mL. In still other embodiments, the target infection cell density is about 5.0×10⁶ viable cells/mL.

In some embodiments, the DOI of the HEK293 cells with the at least one non-replicating adenovirus containing the at least one transgene encoding human interferon is from about 32 hours to about 72 hours. In some embodiments the DOI is from about 48 to about 72 hours. In some embodiments, the DOI is about 32 hours, about 33 hours, about 34 hours, about 35 hours, about 36 hours, about 37 hours, about 38 hours, about 39 hours, about 40 hours, about 41 hours, about 42 hours, about 43 hour, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, or about 72 hours. In yet other embodiments, the DOI is about 48 hours. In yet other embodiments, the DOI is about 72 hours.Before the present disclosure, Nadofaragene firadenovec was produced from cells grown in adherent cultures. It is surprisingly herein discovered that Nadofaragene firadenovec can be produced from suspension cultures of HEK293 cells. Moreover, in some embodiments, when VPC2.0 cells were infected with Nadofaragene firadenovec according to the methods described herein, it was surprisingly discovered that the VPC2.0 cells produce Nadofaragene firadenovec in an amount of at least about 100% to about 500% greater than HEK293 cells (ATCC No. CRL-1575) or HEK293F cells infected with Nadrofaragene firadenovec. In yet other embodiments, when VPC2.0 cells were infected with Nadofaragene firadenovec according to the methods described herein, it was surprisingly discovered that the VPC2.0 cells produce Nadofaragene firadenovec in an amount of at least about 500% to about 575% greater than HEK293 cells (ATCC No. CRL-1575) or HEK293F cells infected with Nadrofaragene firadenovec. In yet other embodiments, when VPC2.0 cells were infected with Nadofaragene firadenovec according to the methods described herein, it was surprisingly discovered that the VPC2.0 cells produce Nadofaragene firadenovec in an amount of at least about 100% greater, at least about 200% greater, at least about 300% greater, at least about 400% greater, at least about 500% greater, at least about 525% greater, at least about 550% greater, or at least about 570% greater than HEK293 cells (ATCC No. CRL-1575) or HEK293F cells infected with Nadrofaragene firadenovec. In yet other embodiments, when VPC2.0 cells were infected with Nadofaragene firadenovec according to the methods described herein, it was surprisingly discovered that the VPC2.0 cells produce Nadofaragene firadenovec in an amount of about 570% greater than HEK293 cells (ATCC No. CRL-1575) or HEK293F cells infected with Nadrofaragene firadenovec.

In yet other embodiments, the cell culture in the bioreactor can be performed in batch, fed batch or perfusion mode. A batch process is a closed system in which a typical growth profile is seen. A lag phase is followed by exponential, stationary and decline phases. In a batch system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. A fed-batch process is still a closed system because cells, products and waste products are not removed. When perfusion is used, the culture medium is at least partially perfused during a portion of time during cell growth of the HEK293 cells (prior to infection) or following infection. Perfusion is used in order to maintain desired levels of certain metabolites and to remove and thereby reduce impurities in the culture medium. Perfusion rates can be measured in various manners, such as in terms of replacement volumes/unit time or in terms of levels of certain metabolites that are desired to be maintained during times of perfusion.

The formation of recombinant adenovirus or recombinant adenovirus vectors takes place inside the HEK293 cells. Thus, the method of the present disclosure comprises harvesting the recombinant adenovirus or recombinant adenovirus vectors from the cells. The recombinant adenovirus or recombinant adenovirus can be harvested from the cells using routine techniques known in the art. For example, in some embodiment, the harvesting can involve a lysis step. In some embodiments, the cell lysis step can be carried out using any conventional or physical means. For example, the cell lysis step can be carried out in the bioreactor by adding at least one lysing agent to the bioreactor. In some embodiments, the lysing agent is a buffer (e.g., in some embodiments, the buffer can contain one or more detergents). In other embodiments, the lysing agent is a detergent. Examples of detergents that can be used include polysorbate (such as polysorbate 20 or polysorbate 80), the Tween® detergents (e.g., Tween®-20 or Tween®-80) or Triton® X-detergents (e.g., Triton® X-100 or NP-40). In some embodiments, the lysis agent is one or more enzymes to the bioreactor. Examples of enzymes that can be used include, for example, nucleases, such as, for example, Benzonase® nuclease, or salt-tolerant enzymes, such as, for example Denerase®, or combinations thereof. In some embodiments, the lysis step involves freezing and thawing the cells using techniques known in the art. In other embodiments, the lysis step involves sonicating the cells. In still other embodiments, the lysis step involves solid shear and/or liquid shear methods.

Once the cells are lysed, this lysed material, which contains the recombinant adenoviruses or recombinant adenovirus vectors, can be collected using routine techniques known in the art to provide the main harvested material. For example, the main harvested material can be obtained by centrifuging the lysed material. In some embodiments, after the cells are lysed, the bioreactor may be rinsed with a conditioning or other buffer to recover as many adenovirus particles from the bioreactor as possible. Thus, in some embodiments, the main harvest material and rinse from the bioreactor can be pooled. This pooling of the main harvested material and the rinse from the bioreactor is collectively referred to as the bulk harvest.

In other embodiments, the recombinant adenovirus or recombinant adenovirus vector is harvested from the cells at least 24 hours after infection. In still other embodiments, the recombinant adenovirus or recombinant adenovirus vector is harvested from the cells at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours after infection.

In some embodiments, once the bulk harvest is obtained, the recombinant adenovirus or recombinant adenovirus vector can be purified from the harvest using routine techniques known in the art. The type of purification is not critical. For example, the bulk harvest and/or the recombinant adenovirus or recombinant adenovirus vector can undergo one or more steps, the steps of clarification, concentration, diafiltration and/or chromatography (Protein A chromatography and/or ion-exchange chromatography).

The following examples are for the purposes of illustration only and are not intended to limit the scope of the claims.

### EXAMPLE 1

### Overview

The focus of this 4×2 L bioreactor study was to demonstrate the bioreactor conditions with the highest viral production of rAd-IFN in the 2 L bioreactor model. In a 4×2 L bioreactor, 1×2 L ROO-01 (HEK-293 adapted to suspension) and 3×2 L VPC2.0 tested with Infection Cell Density, MOI, DOI, and pH range set at 7.2.

### Preculture and Cell Expansion

The process for production of rAd-IFN was initiated by the thaw of one RCB of the Rooster cell line adapted to FujiFilm BalanCD media and one RCB of VPC2.0 in the Viral Production Medium (ThermoFisher Scientific) and subsequent expansion in shake flasks. The cells were cultured through a series of passages at targeted cell densities based on the passage duration. Passaged cells were maintained in incubators operating at 80% humidity at 37° C. in un-baffled Erlenmeyer flasks of varying sizes at 125 rpm with a 25-mm throw for up to 96 hours before subsequent passaging. The CO₂%, orbital throw diameter, and agitation rate are shown in below Table A for each cell line. The production bioreactors were inoculated at targeted seeding densities (see Table B) and cell growth was allowed to proceed for two days prior to infection.

TABLE A

| Parameter | VPC2.0 | ROO |
|---|---|---|
| CO₂ | 8 | 5 |
| Throw (mm) | 25 | 19 |
| Agitation before (rpm) | 125 | 120 |

TABLE B

| | VPC2.0 | | ROO | |
|---|---|---|---|---|
| | Seed VCD | Passage | Seed VCD | Passage |
| | Target | Duration | Target | Duration |
| Passage | (10⁶ VC/mL) | (hours) | (10⁶ VC/mL) | (hours) |
| Thaw - P0 | 0.30 | 96 | 1.0 | 96 |
| P1 - | 0.30 | 96 | 0.75 | 96 |
| Production | 0.60 | 72 | 1.0 | 72 |

### 2 L UniVessel Production Schedule

The production schedule for a 2 L UniVessel is shown Table C below:

TABLE C

| Production Day | Task |
|---|---|
| Day 0 | Inoculation, Cell Culture Sampling |
| Day 1 | Cell Culture Sampling |
| Day 2 | Cell Culture Sampling, Infection |
| 1-Day Post Infection | Cell Culture Sampling |
| 2 Day Post Infection | Cell Culture Sampling, Harvest |

Inoculation (Day 0) Inoculation was performed as follows:
Cell counts were taken from the final passage of seed train, and that count was used to calculate the volume of cells needed to reach the target seeding density.
The required number of cells were collected from that train and transferred aseptically into the bioreactor for production.
A cell culture sample was collected after inoculation to confirm seeding density.

### Infection & Harvest Purified

rAd-IFN virus was used to infect VPC2.0 and ROO cells after cultures were grown for 48 hours. The design for this 2 L bioreactor study is summarized in Table D. Samples were collected at the time of harvest and lysed for 2 hours at 37° C. with 10× Lysis Buffer to a final concentration of 1× to release intracellular rAd-IFN virus. The lysates were analyzed for rAd-IFN titers by digital polymerase chain reaction (dPCR).

TABLE D

| Study | Cell | Number of | Infection | | | pH |
|---|---|---|---|---|---|---|
| Number | Line | Vessels | Density | MOI | DOI | ranges |
| 4 × 2L | ROO | 1 | 1.50E+06 | 200 | 48 | 7.2 ± 0.03 |
| Bioreactor | VPC2.0 | 3 | 3.00E+06 | 175 | | 7.2 ± 0.05 |

The ROO 2 L bioreactors titer average viral titer was 5.54E+10 vg/mL. The VPC2.0 2 L bioreactors' titer average viral titer was 2.96E+11 vg/mL.

### Estimates of Doses Produced Per Batch

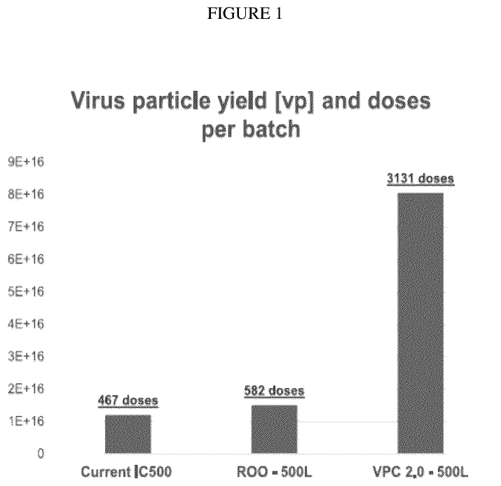

Based on the average viral titer for the ROO 2 L bioreactors and the VPC 2.0 2 L bioreactors, the number of doses of rAd-IFN (ADSTILADRIN®) produced per 500 L batch can estimated by multiplying the titer produced in the bioreactor at 2 L by 250 (i.e. to provide 500 L). One (1) dose of rAd-IFN (ADSTILADRIN®) equals 2.6E+13 vp/mL. As shown in FIG. 1, the estimated number of doses of rAd-IFN (ADSTILADRIN®) that could be produced by the ROO bioreactors would be 582 doses. In contrast, the estimated number of doses of rAd-IFN (ADSTILADRIN®) that would be produced by the VPC2.0 bioreactors would be 3131 doses. FIG. 1 also shows the number of doses of rAd-IFN (ADSTILADRIN®) produced by the currently U.S. FDA approved method which employs HEK293 cells in adherent mode (“Current IC500”). As shown in FIG. 1, the estimated number of doses of rAd-IFN (ADSTILADRIN®) produced using VPC2.0 cells is 570% greater than the using the prior art methods (e.g., Current IC500 and HEK293 cells adapted to suspension).

### EXAMPLE 2

### Cell Expansion

One vial of VPC2.0 cells is thawed into 125 mL shaker flask in 30 mL of Viral Production Medium (available from ThermoFisher) supplemented with 4 mM Glutamax as instructed by the manufacturer (User guide for Viral Production Cells 2.0 and Viral Production Medium). Cells are incubated in +37° C. and 8% CO₂ shaking 125 rpm (19-mm shaking diameter) until 1-3E+06 cells/mL are reached. For routine cell maintenance cells are passaged every 3-4 days with 0.3-1.0E+06 cells/mL (recommended seeding densities 0.3E+06 cells/mL for 4 days and 0.6E+06 cells/mL for 3 days). Passaging should be done only when cell density has reached ≥4E+06 cells/mL.

Confirmation of rAd-IFN Production in VPC2.0 Cells

Cells are expanded in shaker flasks until enough cells are obtained for the test. Cells are counted using Countess or manually using haemocytometer. When sufficient cell number is reached, cells are split to 3×250 mL shaker flasks (60.5 mL per flask) with 0.75E+06 cells/mL. After 24 hours (±2 hours) cells are counted (0.5 mL sample taken from flasks) and infected with rAd-IFN, also known as Nadofaragene firadenovec (secondary Working Viral Seed Stock (sWVSS),, 6.8E+11 vp/mL) using MOI 200. After infection, flasks are incubated in +37° C., 8% CO₂ and 125 rpm shaking. Harvest is performed 48 hours (±2 hours) from infection. Before harvest is started, 3 mL from each shaker flask is taken to separate 15 mL tubes and centrifuged 200×g for 6 minutes. Samples (4×600 μL, 450 μL sample+150 μL QC-buffer (which contains HEPES and glycerol are taken from supernatant and stored below −60° C. Harvest is initiated by adding 10× lysis buffer (final 1×; the lysis buffer contains HEPES, Tween 20 and MgCl₂) and incubating shaker flasks for 2 hours (±10 min; in +37° C., 125 rpm shaking). Part of the lysed material is transferred to 50 mL centrifuge tubes and is centrifuged 1940×g for 20 minutes. 5×600 μL samples (450 μL sample+150 μL QC-buffer) are collected from supernatant and are stored below −60° C. ddPCR is used to analyse the viral genome titers (vg/ml) from samples.

It will be readily apparent to those skilled in the art that other suitable modifications and adaptations of the methods of the present disclosure described herein are readily applicable and appreciable and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure and should not be viewed as limiting to the scope of the disclosure. The disclosures of all journal references, U.S. patents, and publications referred to herein are hereby incorporated by reference in their entireties.

The present disclosure has multiple aspects, illustrated by the non-limiting examples described herein.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A method of manufacturing Nadofaragene firadenovec, the method comprising the steps of:
a) infecting Viral Production Cells 2.0 (VPC2.0 cells) with Nadofaragene firadenovec; and
b) harvesting the Nadofaragene firadenovec produced by the VPC2.0 cells,
wherein the Nadofaragene firadenovec is produced in an amount at least 100% greater than a method in which HEK293 cells are infected with Nadofaragene firadenovec.

2. (canceled)

3. (canceled)

4. (canceled)

5. (canceled)

6. The method of claim 1, wherein the vector is harvested at least 24 hours after infection.

7. The method of claim 1, wherein the vector is harvested at least 36 hours after infection.

8. The method of claim 1, wherein the vector is harvested about 48 hours after infection.

9. The method of claim 1, wherein the VPC2.0 cells are cultured in suspension.

10. The method of claim 1, wherein multiplicity of infection of the vector to VPC2.0 cells is about 100 to about 300.

11. The method of claim 10, wherein multiplicity of infection of the vector to VPC2.0 cells is about 175 to about 200.

12. The method of claim 1, wherein the VPC2.0 cells are inoculated in a bioreactor at a target seeding cell density of about 0.75×10⁶ viable cells/mL.

13. The method of claim 1, wherein the VPC2.0 cells are infected at a target infection cell density of from about 2×10⁶ to about 4×10⁶ viable cells/mL.

14. (canceled)

15. The method of claim 1, wherein the Nadofaragene firadenovec is produced in an amount at least 200% greater, at least 300% greater, at least 400% greater, or at least 500% greater than a method in which HEK293 cells are infected with Nadofaragene firadenovec.

16. The method of claim 1, wherein the method further comprises lysing the VPC2.0 cells with a lysing agent prior to harvesting.

17. The method of claim 16, wherein the method further comprises centrifuging the lysed cells.

18. The method of claim 1, wherein the method further comprises purifying the vector after harvesting.

19. A method of manufacturing Nadofaragene firadenovec, the method comprising the steps of:
a) infecting a clonal cell line derived from HEK293F cells grown in suspension with Nadofaragene firadenovec, wherein: (1) the target infection cell density is from about 1×10⁶ to about 4×10⁶ viable cells/mL; (2) infection is carried out at a multiplicity of infection of about 100 to about 300 and a duration of infection is about 48 hours; and (3) wherein the Nadofaragene firadenovec is produced in an amount at least 100% greater than a method in which HEK293F cells are infected with Nadofaragene firadenovec and
b) harvesting the Nadofaragene firadenovec produced by the clonal cell line.

20. (canceled)

21. (canceled)

22. (canceled)

23. (canceled)

24. (canceled)

25. The method of claim 19, wherein the vector is harvested at least 24 hours, at least 36 hours, or at least 48 hours after infection.

26. The method of claim 19, wherein the cells are cultured in suspension.

27. The method of claim 19, wherein multiplicity of infection of the vector to cells is about 175 to about 200.

28. The method of claim 19, wherein the clonal cell line derived from HEK293F cells are inoculated in a bioreactor at a target seeding cell density of about 0.75×10⁶ viable cells/mL.

29. The method of claim 19, wherein the method further comprises lysing the cells with a lysing agent prior to harvesting.

30. The method of claim 19, wherein the method further comprises purifying the vector after harvesting.
